(19) United States
(12) Patent Application Publication
(10) Pub. No.: US 2019/0336276 A1
(43) Pub. Date: Nov. 7, 2019

(54) Voice prosthesis with disposable inner cannual valve assembly

(71) Applicant: Brian Kamradt, Indianapolis, IN (US)

(72) Inventor: Brian Kamradt, Indianapolis, IN (US)

(73) Assignee: Brian Kamradt, Indianapolis, IN (US)

(21) Appl. No.: 15/932,830

(22) Filed: May 3, 2018

(51) Int. Cl. A61F 2/20

(52) U.S. Cl. CPC A61F 2/20; A61F 2/203; A61F 2240/001; A61F 2220/0025; A61F 2250/0069; A61F 2250/0059; A61F 2250/006

(57) ABSTRACT

A voice prosthesis to be mounted in a fistula between trachea and esophagus includes an outer cannula and a removable, disposable inner cannula. Both outer and inner cannula are tubular shaped with the inner cannula fitting inside the outer cannula. The inner cannula element with a through passage and a valve mechanism controlling the connection through said passage. A coupling mechanism is provided on the proximal end of the inner cannula to provide easy, releasable attachment of the inner cannula to the outer cannula. The inner cannula is inexpensively constructed and is dimensionally compatible with the outer cannula.

### TECHNICAL FIELD

The present invention relates to a voice prosthesis having an outer cannula and a removable inner cannula, and in particular to a voice prosthesis having a disposable inner cannula.

### BACKGROUND OF THE INVENTION

When a person's larynx has been removed by surgery due to pathological changes in the throat, the trachea is sutured to an opening in the throat (tracheostoma). By the surgery, the person has lost the ability to speak, and in order to restore this ability to speak a method has been applied for several years, in a voice prosthesis of the kind referred to above is mounted in a fistula, i.e. a passage between trachea and esophagus. At speech, the tracheostoma is occluded by sealing the same either by the patient placing the fingers against the tracheostoma or by the tracheostoma being closed by an in connection there with provided stoma valve. Then, the expiration air is pressed from the lungs through the voice prosthesis into esophagus where the mucous membranes of the throat are brought into vibration and speech is produced as a consequence thereof. Several voice prostheses are described in U.S. Pat. Nos. 4,911,716, 4,435,853, 4,820,304, and DD-AI-275 183. The voice prosthesis is fixed in the fistula by means of two flanges on the spool-shaped element. The spool-shaped element can be cylindrical or oval and preferably it is made of silicon rubber. All existing voice prostheses have in common that they provide a check valve function, which means that the valve mechanism normally is closed but opens when air is pressed from trachea via the valve to esophagus. The valve mechanism is maintained in the closed position by spring bias which in most cases is maintained by elasticity of the material from which the voice prosthesis is made.

When existing voice prostheses are mounted in the fistula the function is acceptable initially but they have a non-acceptably short life due to growth of fungus, Candida, will cover the sealing surfaces of the valve mechanism and causing an obstruction of the valve which causes leakage at the intake of beverages and is the primary reason for exchange of the voice prosthesis. Also, the fatigue of the spring bias (the material of the voice prosthesis) so that the valve as a consequence thereof will be partly open in the normal position causing leakage through the voice prosthesis.

The following are of interest: U.S. Pat. Nos. 3,693,624; 4,315,505; 5,314,470; 5,578,083; 7,166,128. No representation is intended by this listing that a thorough search of material prior art has been conducted, or that no better art than listed is available.

### SUMMARY OF THE INVENTION

As mentioned above the growth of Candida on the sealing surfaces of the voice prosthesis is the main reason for the necessity of exchanging the voice prosthesis. It is an object of the present invention to provide a voice prostheses with outer cannula, which remains in place mounted in the fistula, and a removable, disposable, and inexpensive inner cannula, which serves as an inner lining of the outer cannula and contains said valve mechanism. Thus, to clear the growth of fungus, Candida, covering the sealing surfaces of the valve mechanism and causing an obstruction of the valve passage way, the inner cannula can be removed, cleaned, or disposed of and then replaced. An important feature of the inner cannula of the present invention, is its disposability. The inner cannula can be quickly and easily produced in large quantities using an extruded molding process and then end-formed to create the flange at the proximal end using a heating process. Furthermore, dimensional tolerances are held close only in the distal area of the inner cannula to assure an adequate seal with the outer cannula.

Further advantageous features of the invention are defined in the dependent claims.

### BRIEF DESCRIPTION OF THE DRAWINGS

FIG. 1 is a representation of the voice prosthesis unit;

FIG. 2 is an exploded view of the voice prosthesis unit;

FIG. 3 is also an exploded view of the voice prosthesis unit;

FIG. 4 is an top view of the voice prosthesis unit;

FIG. 5 is an section view of the voice prosthesis unit;

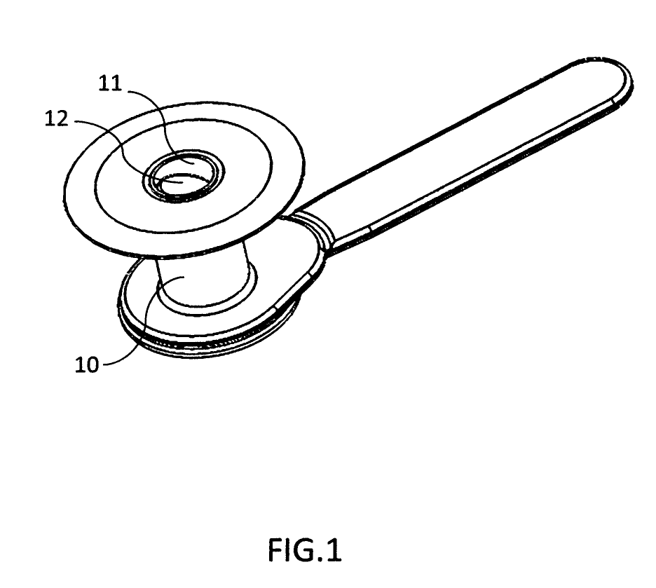

### DETAILED DESCRIPTION

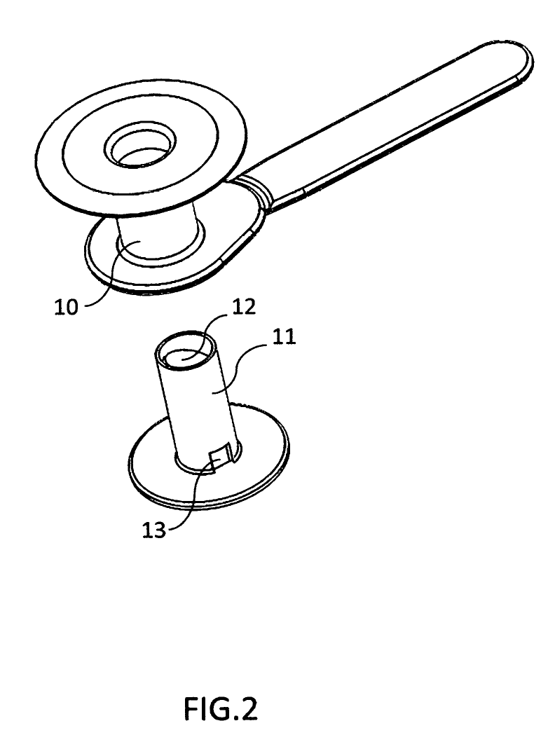

Referring to all FIGS. there is shown the voice prosthesis unit of the present invention, including a tubular outer cannula 10 made of a silicone rubber or a thermal plastic elastomer and a removable tubular inner cannula 11 made of a rigid plastic material, shown removed in FIG. 2.

The outer cannula 10 is for insertion into the fistula, i.e. a passage between trachea and esophagus of the patient through an opening in the neck and has a flange and opening at both distal and proximal ends.

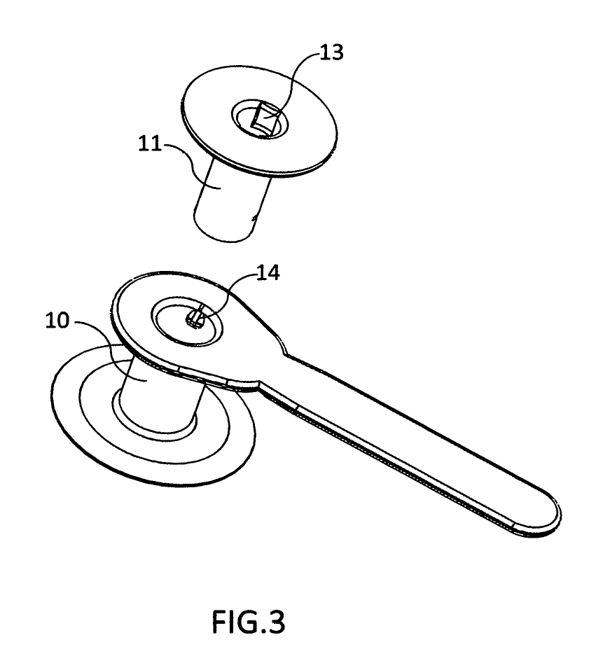

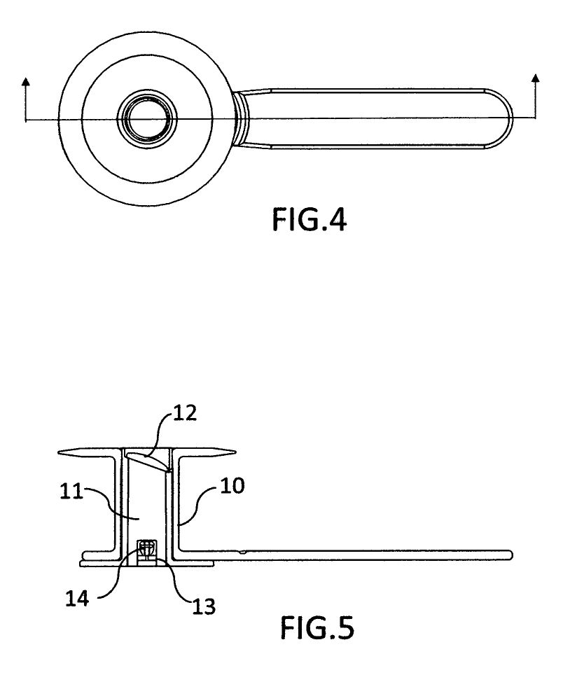

The inner cannula 11 is inserted into the proximal opening of the outer cannula 10 and secured in place by means of a coupling feature located on its proximal end as shown in FIG. 3. The coupling feature is a pair of windows 13 which engage retaining nubs 14 located on the proximal end of the outer cannula 10. The windows 13 can be connected to an insertion tool, not shown.

The outer cannula 10 is made of a compliant material such as silicone rubber or a thermal plastic elastomer material and therefore the retaining nubs 14 of the outer cannula 10 are compliant and will move out of the way as the rigid plastic inner cannula 11 is inserted. The inner cannula 11 has windows 13 that when placed in the desired position within the outer cannula 10 will allow the nubs 14 to spring back into its molded shape. Further the outer cannula has a reduced diameter portion 15 near its esophageal end as shown in FIG. 2. and the inner cannula surface 11 abutting against the reduced diameter portion 15 on the outer cannula when the inner cannula is inserted into the outer cannula forms a gas seal means for preventing leakage of gas from the voice prosthesis.

The inner cannula 11 forms a passageway to permit air to flow from the trachea into the esophagus. FIG. 5 is a sectional view of the voice prosthesis unit of the present invention.

Mounted and located just at the distal end behind the tip of the inner cannula 11 is a flap valve 12.

An important feature of the inner cannula 11 of the present invention, is its disposability, it is relative in expense associated with the polymer materials and methods of manufacture from which it is constructed. For example, the inner cannula 11 can be quickly and easily produced in large quantities using an injection molding or extrusion molding process and then end-formed to create the flange at the proximal end using a heating and forming process.

Furthermore, dimensional tolerances are held close only in the distal area of the inner cannula 11 and outer cannula 10 to assure an adequate seal. This provides for ease of insertion of the inner cannula 11 into the outer cannula 10 and interchangeability of the inner cannula 11.

Furthermore, as described above, these inner cannulas 11 are sufficiently economical in terms of material and manufacturing cost to make their disposability practical. The inner cannula 11 with incorporated flap valve 12 can be removed, cleaned, or disposed of making it easy to replace when in case of an obstructed flap valve 12. Thus, adding to the longevity of the outer cannula 10 and reducing the frequency of replacement needed.

1. A voice prosthesis for insertion into a fistula in a tracheoesophageal wall of a patient, comprising:
an outer cannula comprising a hollow, annular, flexible body configured to be inserted into a fistula and to maintain the fistula open, the outer cannula further comprising a first retention flange at a tracheal end of the body, and a second retention flange at an esophageal end of the body, the body having a through passage defined therein between the tracheal end and the esophageal end; and
a removable inner cannula comprising a hollow, annular, rigid body to be inserted into the through passage of the outer cannula, the inner cannula further comprising a retention flange at a tracheal end of the inner cannula when positioned into the passageway, the rigid body having a through passageway defined therein, the rigid body open at both the tracheal end of the inner cannula and an esophageal end of the inner cannula, the rigid body.

2. The voice prosthesis of claim 1, wherein the outer cannula further comprises retaining nubs for retaining the inner cannula in the outer cannula.

3. The voice prosthesis of claim 7, wherein the inner cannula further defines a recess in an inner wall of the inner cannula, the recess configured for positioning the valve in the recess at an increased diameter portion of the inner cannula on the esophageal end.

4. The voice prosthesis of claim 3, wherein the outer cannula has a reduced diameter portion near the esophageal end of the outer cannula, and wherein a surface of the inner cannula abuts against the reduced diameter portion of the outer cannula when the inner cannula is inserted into the outer cannula.

5. The voice prosthesis of claim 2, wherein the inner cannula has retention windows defined therein, the retention windows configured to receive the retaining nubs of the outer cannula to retain the inner cannula in the outer cannula.

6. The voice prosthesis of claim 5, wherein the retention windows are located at or near the tracheal end of the inner cannula.

7. The voice prosthesis of claim 1, further comprising a valve positioned within the inner cannula at the esophageal end of the inner cannula.

8. The voice prosthesis of claim 7, wherein the valve is positioned within the inner cannula at an increased diameter portion of the inner cannula.

9. The voice prosthesis of claim 4, wherein when the inner cannula surface abuts against the reduced diameter portion, a gas seal is formed, the gas seal capable of preventing a leakage of gas from the voice prosthesis.

10. The voice prosthesis of claim 1, wherein the inner cannula comprises an internal feature configured to receive a valve.

11. The voice prosthesis of claim 1, wherein the inner cannula is disposable.

12. The voice prosthesis of claim 1, wherein the outer cannula comprises a first material, and wherein the inner cannula comprises a second material, the second material more rigid than the first material.

13. The voice prosthesis of claim 1, wherein a length of the outer cannula is approximately equal to a length of the inner cannula.
